(19) United States
(12) Patent Application Publication
(10) Pub. No.: US 2010/0222705 A1
(43) Pub. Date: Sep. 2, 2010

(54) METHOD OF MEASURING QUALITY OF THE EQUINE DISTAL PHALLANGE FROM A LATERAL-MEDIAL RADIOGRAPH

(76) Inventor: John J. Craig, Paso Robles, CA (US)

Correspondence Address: John J. Craig, 6720 Linne Rd, Paso Robles, CA, 93446, US

(21) Appl. No.: 12/395,683

(22) Filed: Mar. 1, 2009

(51) Int. Cl. A61B 5/103

(52) U.S. Cl. 600/587

(57) ABSTRACT

A method to determine the relative concavity of the palmar aspect of the distal phalange of the equid from a standard lateral-medial radiograph. Horses are born with a relatively large concavity, and with age, through a demineralization process, the concavity reduces. The method gives a way to monitor and assess this concavity and can be used in various ways, e.g. as an indicator of future soundness likelihood.

Radiographic assessment of the equine foot has been part of the veterinary evaluation for over 70 years. Multiple radiographic projections are used to thoroughly evaluate the bones of the digit so an opinion can be rendered regarding the presence of pathology, as well as, the nature and severity of that pathology. In the equine digit, remodeling of the distal phalange is an important indicator of the overall health of the foot. We have developed a new method based on the Lateral-Medial (LM) radiograph of the equine digit which allows us to estimate the relative ‘flatness’ or ‘cuppiness’ of the solar surface of the distal phalange (sometimes called the ‘pedal bone’ or the ‘P3’ bone). Our method specifies a way of computing a quantity that we call the ‘palmar-metric’, which gives a measure of the volume of the concavity on the solar or palmar side of this bone. Our studies indicate that this volume tends to decline throughout the life of any given horse due to remodeling (due to demineralization) of the bone. Horses start out life with relatively ‘cuppy’ pedal bones, which gradually flatten out (losing their ‘cuppiness’) as they age. This method of measuring and assessing the state of the equine distal phallage may be helpful in the future to measure the health of the hoof and assess the effect of environmental factors and age on the coffin bone.

A new measure was developed, the “palmar-metric” which is based on measuring the area under a profile we call the ‘palmar curve’ which is evident in a high quality LM radiograph of the equine digit (FIG. 1). This contour is visible due to the increased density of the bone along that aspect due to mechanical needs of the Deep Digital Flexor Tendon (DDFT) that inserts there. Our palmar-metric is a calculated ratio, expressed as a percentage, of the area under the coffin bone relative to the area of a certain rectangle described by the bone (FIGS. 2, 3). Its value is dependent on the shape of the solar surface of the coffin bone. The more curve to the bone (solar concavity) the greater the ratio. Formulating this measure as a ratio of areas means that no scaling system is required for the radiograph, and also that our metric is not dependant on foot size. The value of the palmar metric ranges from zero (a totally flat pedal bone) up to values as high as 20.0 or so.

The method is to view the palmar curve as a mathematical function described relative to a coordinate system located at the distal tip of P3. To be precise, the origin of the reference system is located at the most distal point of the palmar curve. The Y-axis is oriented upwards, and the X-axis points back towards the caudal portion of the P3 bone. For a cadaver bone, its orientation when rested on a flat surface is “vertical” (FIG. 3A). For in-vivo bones, one must make an estimation of the “P3 palmar angle” and then use it to define vertical (FIG. 3B). The “palmar angle” is a popular radiographic measure currently in use by veterinarians when describing the orientation of the coffin bone within the hoof capsule. Several values could be computed from the palmar curve, for example, it's “straightness” ahead of the extensor process, and other measures. However, the measure found most useful has to do with the “area under the curve” for the portion of the palmar curve that lies distal to the perpendicular dropped from the extensor process (FIG. 2). In general, young and/or healthy feet, will have more area under the curve. This measure gives a notion of how “cuppy” or how “flat” the palmar surface of P3 is.

Additionally, when scaled radiographs are available, a secondary and related computation can estimate the physical volume of the concavity of the solar aspect of the pedal bone in cubic centimeters (cc).

In one portion of our investigations, we used scaled radiographs to compute an estimate of the physical volume of the concavity of the solar side of the P3 bone. This computation is related to our palmar-metric, but requires radiographs that can be accurately scaled for length measurements. On 65 cadaver bones we carefully measured the actual volume of the palmar cup as shown in FIG. 4. We then radiographed the bones, traced the palmar-curve, and computed an estimate of the volume. The 65 bones used in the study had an average palmar cup volume of about 11 cc and our average error in estimating this volume from the LM radiograph was about 1 cc. FIG. 5 shows a plot of our estimates, computed from the LM radiograph, compare to the actual, experimentally measured volume. The best fit line has a regression coefficient of 0.958.

As an example of our results, in one study of 278 quarterhorse feet of known age, the palmar-metric was calculated (FIG. 6). The red curve indicates the 3^{rd}-order trend line of the data, and indicates that the palmar-area metric, on average, decreases with age. The wide scatter of the data points indicates that many factors other than age must also affect the palmar metric. One could determine if a given individual lies above or below the value of the trend line for it's age as a means of rating the horse as having a below or above average P3 bone.

We believe that for any animal, once the pedal bone is mature, this palmar-area metric will stay the same or decrease as the animal ages. We believe this metric is a useful means to capture the net effect of how the animal's lifestyle has impacted the quality of the distal phalange. It is hoped that a better understanding of how and why coffin bones remodel throughout the animal's lifetime, along with a way to measure this process from standard radiographs, will yield improvements in hoof care for the horse.

### FIGURE CAPTIONS

FIG. 1: The ‘palmar-curve’ is evident in LM radiogrpahs. It is a bright contour which corresponds to midline of the palmar concavity of the pedal bone.

FIG. 2: Our ‘palmar-metric’ is the number corresponding to the percentage of the area of the rectangle that is under the palmar-curve. For the pedal bone in this radiograph, the value of our metric is 13.4.

FIG. 3: In FIG. 4A, a cadaver bone rests on a horizontal surface. A coordinate system has its origin at the most distal point on the palmar curve. The blue line is constructed as a vertical going through the highest point on the extensor process. In FIG. 4B, a radiograph of P3 within the hoof is referenced in the same way, except that an estimation of the palmar angle must be made, and the coordinate system aligned with it.

FIG. 4: Experimentally measuring the volume of the solar cavity (ahead of the extensor process peak) for 65 cadaver bones. Putty was used to fill the concavity (A); the bone was placed with the extensor process above a marker so that a line was left in the putty (B) indicating where to trim (C). The putty (D) was removed and measured by water displacement to within 0.1 cc. The process was repeated 3 times for each bone and the results averaged.

FIG. 5: In a calculation related to our proposed metric, we can compute an estimate of the physical volume of the solar concavity when a scaled radiograph is available. This figure shows that our estimate of volume matches well the actual volume measured for a group of 65 cadaver bones.

FIG. 6: Results of our study of 105 feet show that the palmar-area tends to decrease with the age of the horse, as shown by the 3^{nd} order trend line shown here in red.

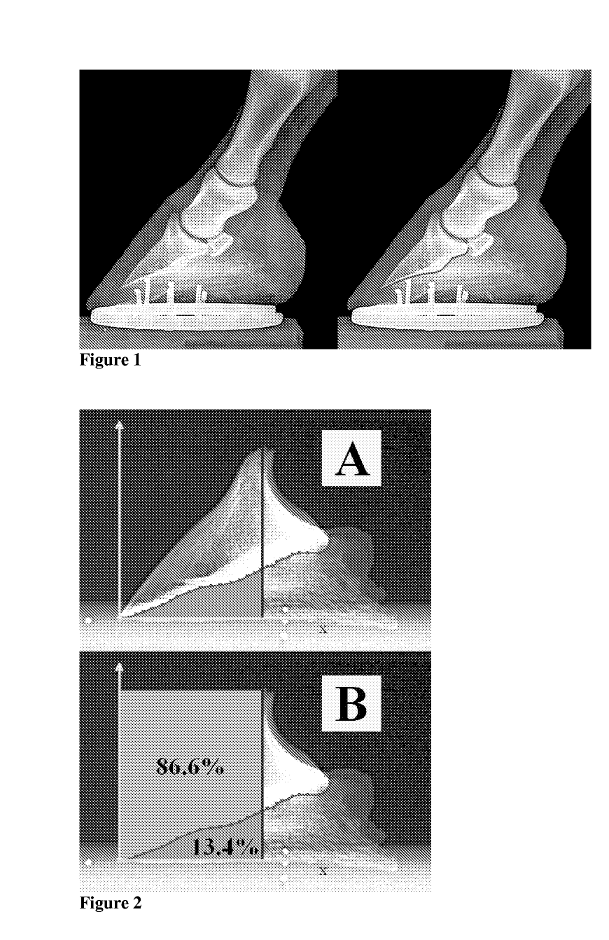

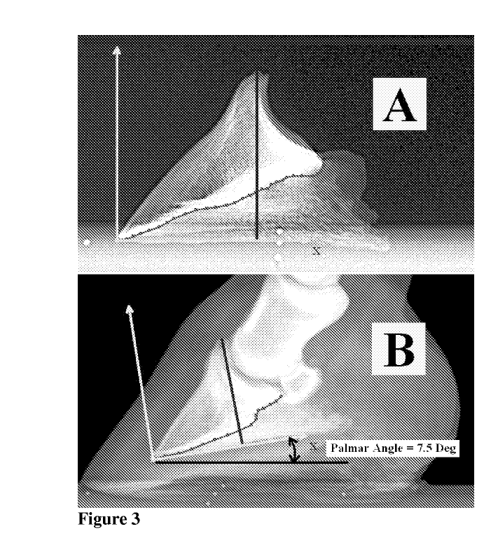

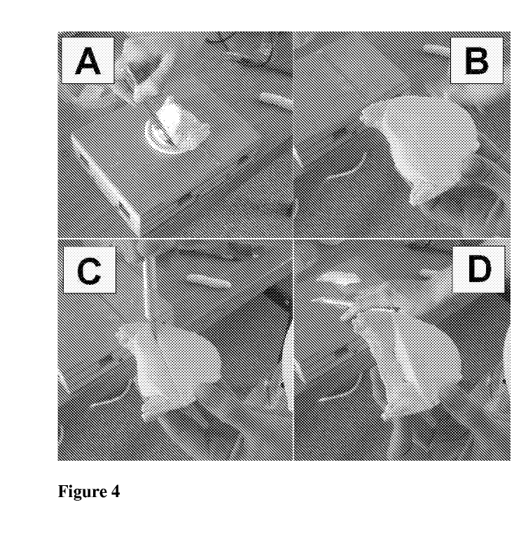

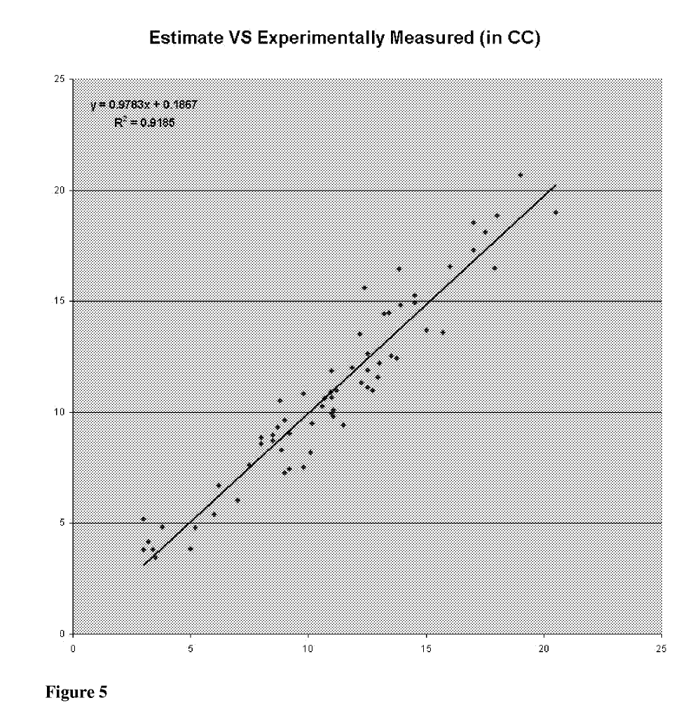

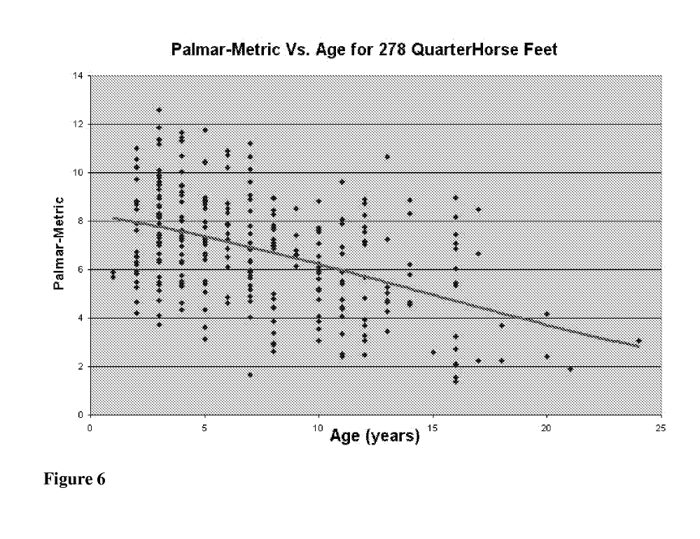

1. A method to compute a dimensionless quantity which gives a measure of the degree of concavity of the palmar surface of the equine distal phalange. This method involves a computation based on points and contours visible in a standard lateral medial (LM) radiograph of the equine foot.

2. A method to compute an estimate of the actual physical volume of the concavity on the palmar side of the equine distal phalange. This computation is based on points and contours visible in a standard lateral medial (LM) radiograph of the equine foot. Said radiograph must contain scaling information in order that our method produce a value in some standard unit of measure (e.g. cubic centimeters).

3. Using the dimensionless quantitative value produced by our method (as in claim 1), a means of making a judgement on the “quality” of the pedal bone. This is done by fitting a trend-line to a large database of such measurements for horses of a certain breed, plotted versus age. Then, to assess a given individual horse, that animal's measurement is compared with this trend line. Above the trendline (i.e. the bone has greater concavity) is a favorable assessment, below it is unfavorable.
